(19) United States
(12) Patent Application Publication
(10) Pub. No.: US 2018/0310928 A1
(43) Pub. Date: Nov. 1, 2018

(54) PNEUMOTHORAX DEVICE

(71) Applicant: THORAXS ISRAEL 17 LTD., Kibbutz Nie-Am (IL)

(72) Inventors: Ariel DRORI, Jerusalem (IL); Liran LEVY, Doar-Na Emek Soreq (IL); Yoav KEN-TOR, Rehovot (IL); Bettina NADORP, Jerusalem (IL); Amnon BUXBOIM, Tel-Aviv (IL); Yaakov NAHMIAS, Rishon-LeZion (IL)

(21) Appl. No.: 15/738,136

(22) PCT Filed: Jun. 21, 2016
(86) PCT No.: PCT/IL2016/050656

§ 371 Date: Dec. 20, 2017

Related U.S. Application Data
(60) Provisional application No. 62/182,589, filed on Jun. 21, 2015.

(51) Int. Cl. A61B 17/02; A61B 90/00; A61B 17/3209

(52) U.S. Cl. CPC A61B 17/0218; A61B 2017/00407; A61B 17/3209; A61B 90/03

(57) ABSTRACT

A method of accessing a thoracic cavity; inserting, in an insertion direction, a thin elongated body, from outside the body, through a space between adjacent ribs, to within the thoracic cavity; expanding the elongated body in a direction including a component perpendicular to the insertion direction to generate an elongated body lumen therein.

### RELATED APPLICATION/S

This application claims the benefit of priority under 35 USC § 119(e) of U.S. Provisional Patent Application No. 62/182,589 filed 21 Jun. 2015, the contents of which are incorporated herein by reference in their entirety.

### FIELD AND BACKGROUND OF THE INVENTION

The present invention, in some embodiments thereof, relates to devices and methods for creating a drainage channel and, more particularly, but not exclusively, to a devices and methods for creating a drainage channel from a pleural cavity e.g. to treat pneumothorax and/or pleural effusion (e.g. hemothorax).

Additional background art includes U.S. Pat. No. 7,811,293, U.S. Pat. No. 6,402,770, U.S. Pat. No. 7,540,875, International Patent Application No. 2014134624, U.S. Pat. No. 7,540,875, U.S. Patent Application No. 20060100657, U.S. Pat. No. 6,517,519, U.S. Pat. No. 7,540,875, U.S. Pat. No. 7,811,293, GB Patent No. GB2495534, U.S. Pat. No. 6,162,236, International Patent Application No. WO2014081810 and International Patent Application No. WO2014081810.

### SUMMARY OF THE INVENTION

According to an aspect of some embodiments of the present invention there is provided a method of accessing a thoracic cavity;

inserting, in an insertion direction, a thin elongated body, from outside the body, through a space between adjacent ribs, to within the thoracic cavity;

expanding the elongated body in a direction including a component perpendicular to the insertion direction to generate an elongated body lumen therein.

According to some embodiments of the invention, the expanding comprises expanding the elongated body until the lumen is suitably sized for drainage from the thoracic cavity without blockage.

According to some embodiments of the invention, the inserting comprises inserting an elongated body which includes a width of at least 1 cm perpendicular to the elongated body long axis and a thickness perpendicular to the elongated body long axis and the width;

wherein a ratio of the width to the thickness is at least 5:1.

According to some embodiments of the invention, the expanding comprises expanding the elongated body thickness.

According to some embodiments of the invention, the method comprises inserting a tube into the lumen.

According to some embodiments of the invention, the method comprises expanding the tube until a lumen of the tube is suitably sized for drainage from the thoracic cavity without blockage.

According to some embodiments of the invention, the method comprises lubricating the tube prior to the inserting of the tube.

According to some embodiments of the invention, the method comprises fixating the tube to patient tissue.

According to some embodiments of the invention, the method comprises removing the elongated body.

According to some embodiments of the invention, the method comprises cutting an incision in a skin surface; and wherein the inserting comprises inserting the elongated element through the incision.

According to some embodiments of the invention, expanding of the elongated body comprises expanding a cross section of more than 80% of an elongated body length simultaneously.

According to some embodiments of the invention, inserting comprises inserting the elongated body to within a pleural cavity.

According to some embodiments of the invention, inserting comprises inserting the elongated body such that an orientation of an elongated body cross sectional long axis is orientated within 10° of a long axis of each of the adjacent ribs.

According to some embodiments of the invention, expanding comprises applying a force to a handle, which force is transferred by an expander into an expanding force which expands the elongated element.

According to some embodiments of the invention, expanding comprises:
applying a force to a handle coupled to the elongated body;
releasing the force; and
reapplying a force to the handle;

wherein the releasing and the reapplying are carried out at least one time.

According to some embodiments of the invention, the method comprises draining excess material from the thoracic cavity.

According to some embodiments of the invention, the method is a method of treatment of pneumothorax and the excess material includes air.

According to some embodiments of the invention, the method is a method of treatment of pleural effusion and the excess material includes liquid.

According to some embodiments of the invention, the excess material comprises one or more of blood, air, fat, body tissue, bone fragments.

According to some embodiments of the invention, the lumen has a rectangular cross section.

According to some embodiments of the invention, the method comprises inserting a device into the lumen.

According to some embodiments of the invention, the device is an imaging device.

According to an aspect of some embodiments of the present invention there is provided a device for establishing a channel to a thoracic cavity from outside a body comprising:

an elongate expandable body, at least 2 cm long, configured to have collapsed state and an expanded state, the body in the expanded state comprising a lumen running through a length of the expandable body;

an expander which transfers a force applied to a handle into an expanding force;

wherein a thickness of the expandable body in the collapsed state is less that 3 mm over the length of the expandable body;

wherein the thickness of the expandable body in the expanded state is at most 2 cm;

wherein the body is expandable from the collapsed state to the expanded state under a resistive force of at least 150N.

According to some embodiments of the invention, a cross section of the body perpendicular to a long axis of the body includes an aspect ratio of at least 5:1.

According to some embodiments of the invention, the device forms part of a kit, the kit comprising a tube sized to fit into the lumen.

According to some embodiments of the invention, the tube is lubricated.

According to some embodiments of the invention, a lumen of the elongate is lubricated.

According to some embodiments of the invention, the handle comprises a first part which moves relative to a second handle part, the handle parts interconnected by a mechanical gain component which transfers force applied to the handle parts to the expander.

According to some embodiments of the invention, the at least one mechanical gain element comprises a lever.

According to some embodiments of the invention, the at least one mechanical gain element comprises a gear.

According to some embodiments of the invention, a ratchet connects the expander and the expandable body; wherein the ratchet prevents retraction of the expander.

According to some embodiments of the invention, a ratchet connects at least one handle portion to the body; wherein the ratchet prevents retraction of the handle.

According to some embodiments of the invention, the device comprises an elastic element coupled to the expander, which elastic element is elastically deformed when the device is in the collapsed state and elastically relaxes applying an expanding force to the expander.

According to some embodiments of the invention, the body comprises a plurality of elongated elements;

wherein the expander comprises least one rigid element coupling the elongated elements;

wherein the expanding force moves the elongated elements with respect to each other;

wherein the at least one rigid element transfers a direction of the force of the elongated elements moving with respect to each other into an expanding force: the movement of the elongated elements changing an angle of the at least one rigid element with respect to the elongated elements, expanding a separation between the elongated elements.

According to some embodiments of the invention, the body comprises two elongated elements, the elongated elements orientated with parallel long axes;

wherein the expanding force moves the elongated elements with respect to each other in a direction parallel to the long axes of the elements.

According to some embodiments of the invention, the body includes a sharp tip positioned for penetrating the chest outer surface.

According to some embodiments of the invention, the sharp tip is retractable.

According to some embodiments of the invention, the device comprises a cover covering an opening of the lumen.

According to some embodiments of the invention, the device comprises a stopper attached to or disposed on the expandable body and increasing a geometry of the expandable body in a direction of expansion of the expandable body, the stopper resisting insertion of the expandable body into the thoracic deeper than the location of the stopper of the body.

According to some embodiments of the invention, the device comprises a plurality of stoppers, where one or more stopper is collapsible into a state where the stopper protrudes from the expandable body by at most 3 mm.

According to an aspect of some embodiments of the present invention there is provided a method of accessing an abdominal cavity;

inserting, in an insertion direction, a thin elongated body from an outer chest surface to within the thoracic cavity between adjacent ribs;

expanding elongated body in a direction including a component perpendicular to insertion direction to generate an elongated body lumen therein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of a method of treatment, according to some embodiments of the invention;

FIGS. 1B-F show a cross sectional view of a device body inserted between adjacent ribs, according to some embodiments of the invention;

FIG. 2A is a simplified side view of an exemplary device, in a closed state, according to some embodiments of the invention;

FIG. 2B is a simplified side view of an exemplary device, in an expanded state, according to some embodiments of the invention;

FIG. 2C is a simplified side view of an exemplary device, after insertion of a tube, according to some embodiments of the invention;

FIG. 2D is a simplified top view of a proximal end of an exemplary device, after insertion of a tube, according to some embodiments of the invention;

FIG. 3A is a simplified schematic top view of exemplary elongated element tips, according to some embodiments of the invention;

FIG. 3B is a simplified schematic side view of exemplary elongated element tips, according to some embodiments of the invention;

FIG. 4A is a simplified schematic side view of a portion of a device including a knife incising a skin surface, according to some embodiments of the invention;

FIG. 4B is a simplified schematic side view of a portion of a device including a retractable knife after penetration by the device of a skin surface, according to some embodiments of the invention;

FIGS. 5A-5D are exemplary embodiments of a connection of a rigid portion to elongated elements, according to some embodiments of the invention;

FIG. 6 is a simplified top view of exemplary shapes for rigid portions for connecting more than one elongated element, according to some embodiments of the invention;

FIG. 7 is a simplified side view of exemplary pluralities of rigid portions, according to some embodiments of the invention;

FIG. 8 is a simplified side view of an exemplary portion of a body of a device including cylindrical shaped rigid portions, in an expanded state, according to some embodiments of the invention;

FIG. 9 is a simplified schematic side view of a device including rigid portion connecting elements, according to some embodiments of the invention;

FIGS. 10A-B are side views of a device including a ratchet mechanism which prevents retraction of a handle portion, according to some embodiments of the invention;

FIG. 11 is a simplified schematic side view of a portion of a body of a device including more than two elongated elements, according to some embodiments of the invention;

FIG. 12A is a side view of a prototype device, in a closed state, according to some embodiments of the invention;

FIG. 12B is a side view of a prototype device, in an expanded state, according to some embodiments of the invention;

FIG. 12C is a side view of a prototype device, after insertion of a tube, according to some embodiments of the invention;

FIG. 12D is a simplified schematic side view of a rigid element, according to some embodiments of the invention;

FIG. 13A is an illustration of an outside view of a pig when a device, in a closed state, is positioned at an incision site on the torso of a euthanized pig, according to some embodiments of the invention;

FIG. 13B is an illustration of an outside view of the pig when the device, in a closed state, has been inserted sufficiently to penetrate the pleural cavity, according to some embodiments of the invention;

FIG. 13C is an illustration of an outside view of the pig after opening of the device, according to some embodiments of the invention; and

FIG. 14 is a simplified schematic side view of an exemplary device including stoppers, according to some embodiments of the invention, according to some embodiments of the invention.

### DESCRIPTION OF SPECIFIC EMBODIMENTS OF THE INVENTION

The present invention, in some embodiments thereof, relates to devices and methods for creating a drainage channel and, more particularly, but not exclusively, to a devices and methods for creating a drainage channel from a pleural cavity e.g. to treat pneumothorax and/or pleural effusion (e.g. hemothorax).

### Overview

A broad aspect of some embodiments of the invention relates to rapidly accessing a thoracic cavity. In some embodiments, access is to establish a drainage channel between the thoracic cavity and outside a body, where the channel is sized sufficiently in cross section such that the channel does not become easily blocked. In an exemplary embodiment, the channel is between a patient pleural cavity and a chest outer surface, through a space between adjacent ribs, for example, for treatment of pneumothorax and/or pleural effusion (e.g. hemothorax).

In some embodiments, an elongate body is inserted into a patient to a desired depth, establishing a channel length, the inserted elongate body, for example, extending from a patient outer chest surface towards a patient thoracic cavity. In some embodiments, the elongate body is then expanded in at least one dimension (e.g. perpendicular to the channel length), generating and/or enlarging a lumen within the elongate body. In some embodiments, the elongate body is expanded sufficiently such that the lumen within the elongate body has a large enough cross section for drainage without becoming blocked (e.g. with blood clots).

An aspect of some embodiments of the invention relates to first establishing a channel length and/or width (perpendicular to the channel length), for example, by inserting a thin, wide, elongate body. The device is then expanded by expanding a thickness of the elongate body, where the thickness is a dimension perpendicular to both the elongate body length and width. In some embodiments, a thin wide elongate body includes a body cross section (perpendicular to a body long axis) with an aspect ratio of at least 2:1, or 5:1, or at least 10:1.

An aspect of some embodiments of the invention relates to inserting a drainage tube, once a lumen with a device body is generated and/or enlarged. Optionally, the elongated body is then removed.

In some embodiments, a thin wide elongate body is inserted between adjacent ribs such that the orientation of the body width is substantially parallel (e.g. within 20°, or 10°, or 5°, or lower, or higher or intermediate angles, of a long axis of one or both ribs) to long axes of the ribs. In some embodiments, ribs resist and/or prevent over-expansion of the elongate body thickness. In some embodiments, an elongate body includes a thin wide portion, for example, which portion is inserted into the patient.

In an exemplary embodiment, the elongate body before expansion and/or the expanded body lumen include a rectangular cross section.

In some embodiments, most of a length of the body lumen (e.g. more than 30%, or more than 50%, or more than 80%, or more than 90% or lower or higher or intermediate percentages) is expanded simultaneously (e.g. within 0.5. second, or 1 second, or within 5 seconds, or longer or shorter or intermediate times).

In some embodiments, an expanding force is applied to the body to expand the body. In some embodiments, an expander to which the expanding force is indirectly or directly applied expands the body. In some embodiments, the expanding force is manually applied by a user e.g. a user applies a force to a handle coupled to the elongate body. Optionally, the force is multiplied by one or more mechanical gain element, for example lever/s and/or gear/s.

In some embodiments, the expanding force is applied in discrete portions, for example, using one or more ratchet mechanism which prevents retraction of the expander (e.g. expanding mechanism) and/or the handle.

In some embodiments, the expanding force is sufficiently large, and/or the device is sufficiently rigid such that the elongate body exerts upon surrounding tissue an expanding force of at least 50-300N, or 100-200N, or 150-200N, or higher or lower, or intermediate ranges or forces.

Additionally or alternatively, in some embodiments, expanding force is applied using pneumatic and/or electrical and/or elastic force. For example, in some embodiments, a power source (e.g. battery), and a motor (and optionally a gear) are used to expand the elongate body. For example, in some embodiments, a balloon is inflated within the elongate body to expand the elongate body.

In some embodiments, the body includes a sharp edge (e.g. a knife), for example, for cutting an initial incision into which the elongate body is inserted. Optionally, the knife is retractable, and/or removable.

In some embodiments, a cover is positioned over the elongate body, for example, covering the lumen of the elongate body, potentially shielding a user from material escaping (e.g. under pressure) from the patient e.g. once the elongate body lumen is opened. In some embodiments, the cover is in place before insertion of the elongate body. Alternatively or additionally, in some embodiments, a cover is positioned after the elongate body is inserted and before the elongate body is expanded.

In some embodiments, the device includes a sealing element and/or valve, for example, positioned within the elongate body, potentially preventing spraying of blood and/or other fluids onto a user during and/or after insertion of the elongate body.

In an exemplary embodiment, a device for establishing a channel includes two elongated elements, where a separation between the elongated elements is enlarged, enlarging a lumen therebetween. In an exemplary embodiment, the two elongated elements are coupled by one or more rigid element. In some embodiments, the expanding force moves the two elongated elements with respect to each other (e.g. in a direction parallel to the elongated element long axes). For example, in an exemplary embodiment, handle portions, one coupled to each elongated elements are moved together, moving the elongated elements with respect to each other. In some embodiments, the movement causes the rigid elements to push the elongated elements apart in a direction perpendicular to the elongated elements long axes.

In some embodiments, the device includes one or more elastic element. In some embodiments, at least one elastic element is elastically deformed when the elongate body is in a collapsed state and, for example, as the elastic element relaxes, it applies an expanding force to said elongate body.

An aspect of some embodiments of the invention relates to rapidly accessing a body cavity (e.g. pleural cavity). In some embodiments, the device is potentially rapidly inserted due to a device geometry. In some embodiments, an elongate element is inserted to a desired depth, and insertion is potentially rapid as the elongate element is thin, reducing the force required to insert the element as compared with an element with larger cross section. Additionally or alternatively, in some embodiments, the device is potentially safely rapidly inserted as device geometry and/or a stopper/s and/or markings on the device enable to rapidly insert the device to a desired and/or non-damaging depth. In some embodiments, depth of insertion is controlled (e.g. by increasing of the geometry of the device along the elongated elements, meaning that insertion decelerates under the same force as the depth increases, e.g. user control via inspection of markings on the device). In some embodiments, depth of insertion is limited, for example, by geometry of the elongated elements, for example, by a stopper element preventing insertion above a certain depth. Insertion is potentially rapid, as a length of a portion of the elongate element (e.g. a thin portion) and/or marking/s on the elongate element and/or a stopper element preventing insertion of the device above a certain depth, meaning a user can easily avoid over-insertion of the device (e.g. over-insertion associated with the device impinging on internal organ/s e.g. the lungs). Once the channel is established, in some embodiments, expansion of the channel to a suitable size, is achieved in a (e.g. rapid) single stage.

An aspect of some embodiments of the invention relates to establishing access to a body cavity (e.g. pleural cavity) without risking damage to internal organ/s (e.g. lungs and/or heart and/or liver, and/or other organs within the thoracic and/or abdominal cavity). In some embodiments, a device for accessing the thoracic cavity includes a blunt and/or curved shape (e.g. a blunt and/or curved distal end), potentially preventing insertion of the device from damaging internal tissue (e.g. organ/s). In some embodiments, the device includes a portion (e.g. an elongated body) full insertion of which is to a depth sized such that the inserted device does not impinge on internal organ/s. In some embodiments, the device includes marking/s and/or a stopper element, potentially preventing over-insertion of the device.

An aspect of some embodiments of the invention relates to using a soft and/or flexible element for drainage and/or access to a body cavity. For example, in some embodiments, a tube is inserted into an elongate body lumen where the elongate body components support the lumen meaning that.

An aspect of some embodiments of the invention relates to accessing an abdominal cavity. In some embodiments, a device elongate body suitable for accessing the abdominal cavity is shorter (e.g. 1-5 cm, 1-3 cm, 1-2 cm shorter) than that of a device suitable for accessing a thoracic cavity. In some embodiments, a device suitable for accessing an abdominal cavity applies less force (e.g. 10-100N, 10-50N).

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Method of Treatment

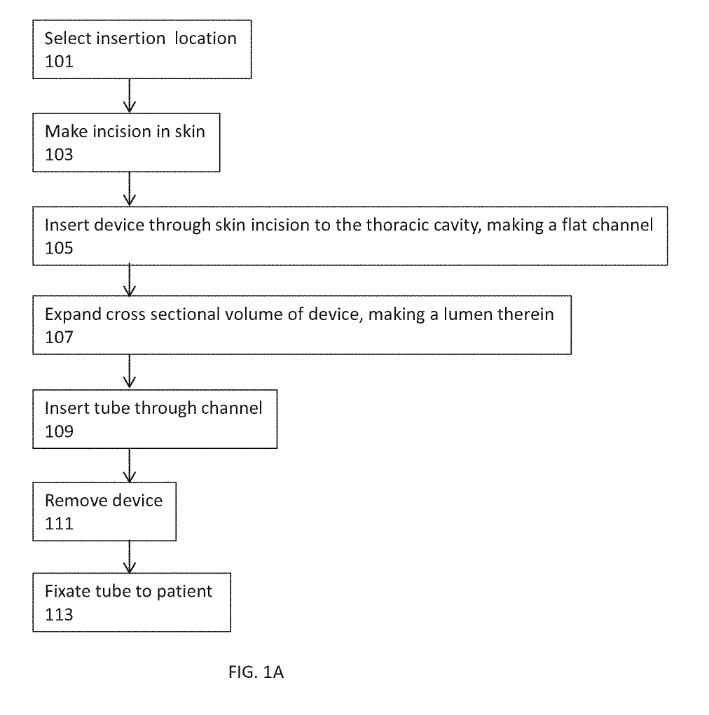

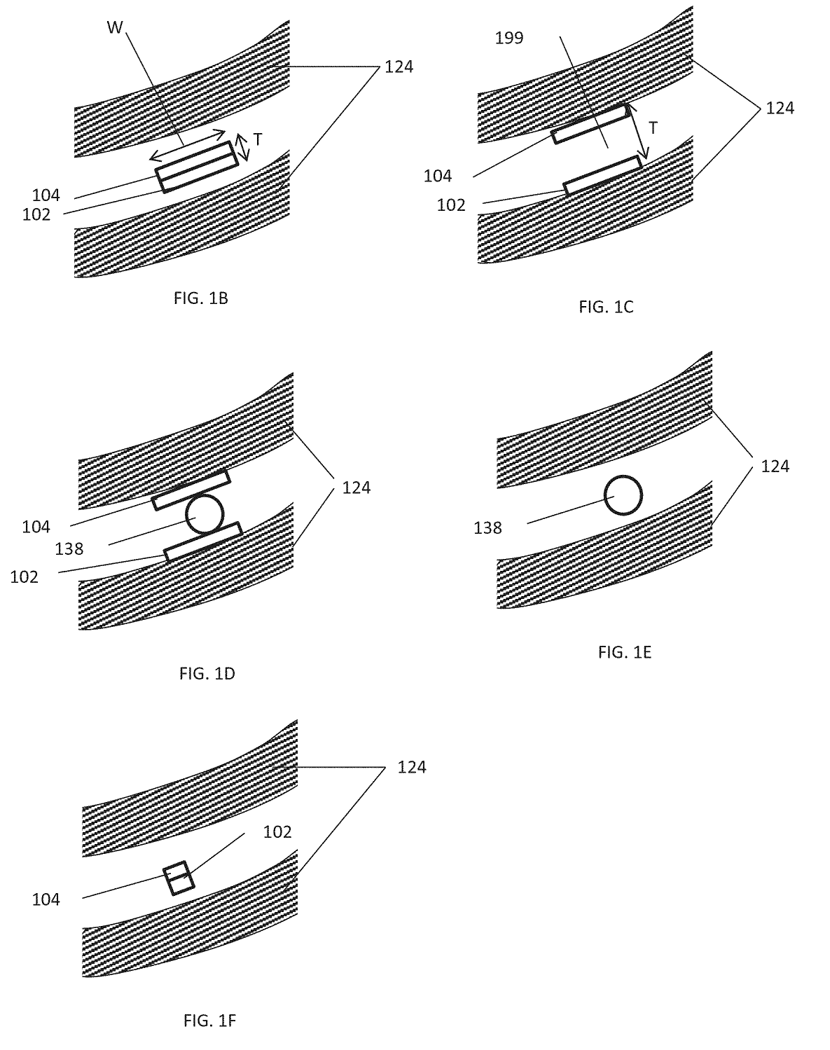

FIG. 1A is a flow chart of a method of treatment, according to some embodiments of the invention.

In some embodiments, treatment includes accessing an area and/or organ behind the ribs (e.g. within the thoracic cavity), access, for example, to organ/s inside the thoracic cavity for treatment (e.g. surgery) and/or imaging (e.g. access is for insertion of an endoscope).

In some embodiments, treatment includes accessing an area within an abdominal cavity.

In some embodiments, treatment is of pneumothorax and/or pleural effusion (e.g. hemothorax) where treatment includes establishing a channel from outside the patient (e.g. an outside surface of the patient's body) to a pleural cavity. In some embodiments, the channel is sufficiently large in cross section to allow flow of fluid and/or gas (e.g. air and/or blood) through the channel, without the channel becoming blocked (e.g. by blood and/or mucus).

At 101, in some embodiments, a location for creation of the channel is selected, for example, by the physician locating an area of the outer chest surface in between two ribs. In some embodiments, an orientation of the channel is selected.

At 103, in some embodiments, an initial incision is made in a patient's skin surface at a selected starting point of the channel. For example, an incision of 1-3 cm length and 0.1-1 cm in depth. In some embodiments, a kit includes a scalpel for making the initial incision.

At 105, in some embodiments, a device body is inserted through the incision and between adjacent ribs to a desired depth, e.g. a depth where a device body tip is within and/or is within a desired separation of a patient's pleural cavity. In some embodiments, the device includes one or more indication of depth, for example, one or more marking on a surface of the body. In some embodiments, a user views the depth indication (e.g. marking/s) to ascertain that the device has been inserted to the desired depth.

Additionally or alternatively, in some embodiments, a device includes an element which prevents a user from over-inserting the device.

In some embodiments, the device includes a change in geometry (e.g. device body is thin for a certain length e.g. device body includes a thin portion which is 1-10 cm or 1-7 cm or 2-5 cm or, for example, in the case of an obese patient, by 10-15 cm) potentially assisting a user in inserting the device body to a desired depth and/or preventing the user from over-inserting the device. In some embodiments, a device body includes a stopper e.g. coupled to the device body and/or handle portion/s, for example, assisting a user in inserting the device body to a desired depth and/or preventing the user from over-inserting the device.

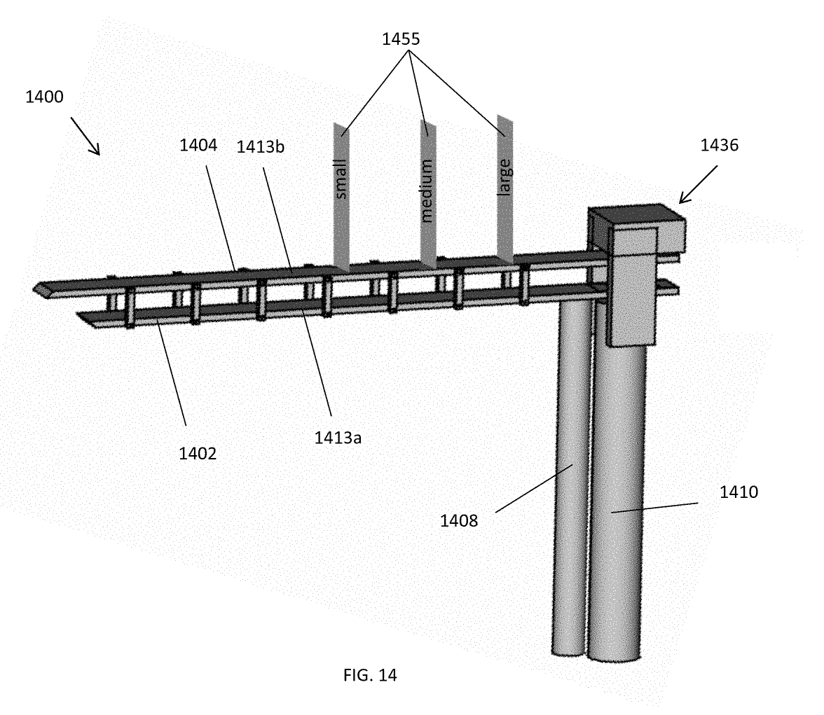

FIG. 14 is a simplified schematic side view of an exemplary device 1400 including stoppers 1455, according to some embodiments of the invention. In some embodiments, a device includes a single stopper. In some embodiments, a device includes a plurality of stoppers 1455, for example, each stopper assisting and/or indicating insertion of the device to a different depth and/or preventing a user from inserting the device more than a certain depth.

For example, different stoppers for different types of patient, exemplary types being child (shallow insertion), adult, average weight, obese (deep insertion). In some embodiments, each stopper has an indication e.g. a written indication, of the usage of the stopper, for example, in some embodiments, e.g. as illustrated in FIG. 14, a patient type is indicated on each stopper.

In some embodiments, an insertion depth is indicated on the stopper. In some embodiments, stoppers 1455 which are not in use do not reduce ease of insertion and/or removal of the device from patient tissue. For example, in some embodiments, stoppers 1455 fold back and/or recess onto elongated element 1404. In some embodiments, once a stopper is folded back and/or recessed onto an elongated element the stopper minimally protrudes from a body of said device, where minimally protruding is defined as protruding by at most by 0.1-3 mm, or 0.1-1 mm, or higher or lower or intermediate values or ranges For example, when the “large” stopper is in use, the “small” and “medium” stoppers fold (e.g. about a hinge attachment to the elongated element) onto elongated element 1404. Alternatively or additionally, in some embodiments, a user removes one or more stopper, for example, in some embodiments, stopper/s include snap fit attachment to the device. Alternatively or additionally, in some embodiments, a user selects a position of a stopper and/or adjusts a position of a stopper before using the device, e.g. the device includes a slide socket (e.g. on the upper elongated element) and the stopper slides to different positions within the slide socket.

In some embodiments, the device is inserted such that a distal end of the device enters the plural cavity. In some embodiments, the device enters the pleural cavity by no more than 3 cm, or 1 cm, or 5 mm, or 2 mm, or 1 mm, or larger or smaller or intermediate values or ranges.

In some embodiments, a device body is inserted by 1-10 cm or 1-7 cm or 2-5 cm or, for example, in the case of an obese patient, by 10-15 cm. In some embodiments, the device body is 10-20 cm long or 5-15 cm long, or 5-10 cm long, or at least 5 cm long, or at least 10 cm long or at least 15 cm long or larger or smaller or intermediate ranges or lengths.

In some embodiments, a kit (e.g. pneumothorax treatment kit) includes one or more device, for example, the kit includes devices with different sized elongated elements (e.g. a device for average patients, and a device for obese patients).

Optionally, in some embodiments, the incision is made during insertion of the device (e.g. the device body includes a sharp leading edge and/or tip). In some embodiments, a device includes a sharp edge (e.g. a knife) which retracts upon establishing the initial incision e.g. after the device is inserted to a certain depth and/or a user retracts (e.g. manually) the sharp edge, potentially preventing the sharp edge from damaging internal areas, e.g. lungs, and/or other internal organs.

At 107, in some embodiments, a lumen is generated (e.g. device is moved from a closed to an open position) between device portions and/or a cross section of a lumen of the device is enlarged.

In some embodiments, the enlarged lumen is sufficiently large to accommodate a tube of diameter 1 cm. In some embodiments, the enlarged lumen is 0.5-2 cm wide, or 0.8-1.5 cm wide, or lower or higher or intermediate ranges or widths. In some embodiments, the enlarged lumen is 0.5-2 cm thick, or 0.8-1.5 cm thick, or lower or higher or intermediate ranges or thicknesses.

FIGS. 1B-F show a cross sectional view of a device body inserted between adjacent ribs 124, according to some embodiments of the invention.

Referring to FIG. 1B, in some embodiments, elongate portions 102, 104 are inserted between adjacent ribs 124 such that the orientation of the body width, W, is substantially parallel to long axes of the ribs. In some embodiments, a device thickness, T is then expanded, as illustrated by FIG. 1C, generating and/or expanding a lumen 199 within the device.

As illustrated in FIG. 1D, in some embodiments, after expansion, a tube 138 is inserted into a lumen within the device. In some embodiments, as illustrated by FIG. 1E, after insertion of the tube, the device body is removed.

Optionally, in some embodiments, a device body is expanded in two directions after insertion. For example, a device is inserted, as illustrated in FIG. 1F, the device is then expanded to a state as illustrated in FIG. 1C.

Potentially, ribs resist and/or prevent over-expansion of the device elongate body thickness. Alternatively, in some embodiments, a device is expandable to a maximum dimension (e.g. thickness), for example, potentially avoiding damaging the ribs and/or making a larger than necessary opening.

In some embodiment, a portion of a device elongate body and/or opening mechanism is reinforced, e.g. thickened. For example, in some embodiments, portion/s of a device elongate body and/or one or more rigid elements (e.g. 206, 506, and/or as described herein) which are, for example, located at the ribs when the device is inserted are reinforced (e.g. made of more robust material, and/or are larger in one or more dimension).

In some embodiments, for example, when the body of the device is inserted into a patient such that an orientation of the body width is substantially parallel to long axes of the ribs the device is expandable to a maximum dimension such that the body does not contact the ribs and/or does not apply significant force to the ribs.

In some examples, the device body is expandable to a maximum dimension perpendicular to a long axis of the device body (e.g. maximum device body thickness) of 0.5-5 cm, or 1-2 cm, or about 1.4 cm, or lower or higher or intermediate ranges or values.

In some embodiments, once the device is expanded, it is locked in position (e.g. using one or more locking element), for example, to prevent the device from closing during insertion of the tube. For example, in some embodiments, the device includes a locking pin. Alternatively, in some embodiments, the device is locked closed (e.g. using a locking element, e.g. a locking pin) and upon release, opens (e.g. under elastic relaxation force).

In some embodiments, the device is closed (e.g. un-expanded) after expanding, for example, to allow re-positioning of the device and/or to ease removal of the device.

Returning back now to FIG. 1A, at 109, in some embodiments, a drainage tube is inserted through the lumen within the device. In some embodiments, fluid and/or gas (e.g. air and/or blood) within the pleural cavity flows out of the pleural cavity through the drainage tube. Potentially, e.g. as the tube is inserted into a lumen, the tube is a flexible and/or soft tube.

In some embodiments, the drainage tube is sufficiently large in cross section to prevent blockage of the tube (e.g. by blood clots and/or mucus). In an exemplary embodiment, the tube includes a 1 cm inner diameter. In some embodiments, a tube inner diameter is 0.5-3 cm, or 0.7-1.5 cm, or 0.8-1 cm, or lower or higher or intermediate values or ranges. In some embodiment a tube outer diameter is 0.5-3 cm, or 0.7-1.5 cm, or lower or higher or intermediate values or ranges. In some embodiments, a tube wall thickness is 0.1-5 mm, or 0.1-1 mm, or lower or higher or intermediate values or ranges.

Exemplary tube materials include plastics, elastomer, composite materials, and silicone rubber. In some embodiments, the tube is flexible and/or soft, potentially preventing discomfort and/or damage to a patient e.g. during movement. In some embodiments, the tube is reinforced (e.g. to prevent kinking and/or closing). In some embodiments, the tube includes a reinforced region (e.g. the tube is constructed of thicker material and/or includes a different material and/or an additional portion) which is positioned were the tube passes between the ribs.

In some embodiments, the tube is and/or elongated elements are coated in lubricant, for example, the tube is provided as part of a kit where the tube is pre-coated in lubricant, potentially easing insertion of the tube. Alternatively, or additionally, a user, before inserting the tube applies lubricant to the tube and/or elongate body lumen. In some embodiments, portions forming the lumen of the device are pre-coated in lubricant and/or a user applies lubricant before inserting the tube.

In some embodiments, one or more of the tube and/or elongate body includes a sheath (e.g. an evert sheath). For example, an evert sheath on an outside of the body, potentially eases (e.g. reduces the force required for) insertion of the body into a patient. For example, an evert sheath on the tube potentially eases (e.g. reduces the force required for) insertion of the tube into the body lumen.

In some embodiments, the tube includes a valve (e.g. a flutter valve) to prevent flow of fluid and/or gas into the pleural cavity.

In some embodiments, a device body includes a collapsed tube (e.g. inserted with the elongate body) and/or a collapsed tube is inserted into the device lumen (e.g. before and/or during expansion of the device). In an exemplary embodiment, the tube includes an internal spring expandable structure.

In some embodiments, a tube includes outer texture and/or protrusion/s (e.g. reverse arrow head shapes) potentially preventing the tube from moving and/or falling out and/or being withdrawn (e.g. during withdrawal of the device body from the patient's body).

At 111, in some embodiments, the device body is removed from the patient's body, leaving the inserted tube in situ.

At 113, in some embodiments, the drainage tube is fixated and/or attached to the patient (e.g. to prevent movement and/or falling out of the tube).

In some embodiments, the tube includes one or more protruding structures, for example, one or more loops and/or ears. In some embodiments, the tube is fixated to the patient by using the protrusion/s e.g. passing a thread through the protrusion and then around the patient's torso and/or stitching the protrusions to the patient tissue. In an exemplary embodiment, a tube includes a plurality of protrusions dispersed along a length of a proximal portion of the tube, potentially enabling fixation of the tube to a patient using these protrusions, for different patient anatomies and/or procedures (e.g. different patients have different depths between the outer chest surface and the pleural cavity, e.g. the tube accesses different portions of the thoracic cavity).

In some embodiments, the tube is fixated by closing the outer opening (e.g. at the outer skin surface of the chest) of the channel around the tube. In an exemplary embodiment, suture/s are used (e.g. a single stitch) to hold the tube. Additionally or alternatively, in some embodiments the tube is secured using additional components, e.g. the tube is taped and/or glued to the patient's outer chest surface. Optionally, the tube is fixated before the device body is removed from the patient, for example, to prevent removal of the device body from dislodging and/or moving the tube.

In some embodiments, a tube is not used, for example, the device is inserted, then expanded and at least a body of the device is left in situ, e.g. providing drainage from and/or access to a body cavity (e.g. to the thoracic cavity). In some embodiments, a portion of the device is removed after expansion, for example, handle portion/s are removed (e.g. unscrewed) from the elongate body.

### Exemplary Devices

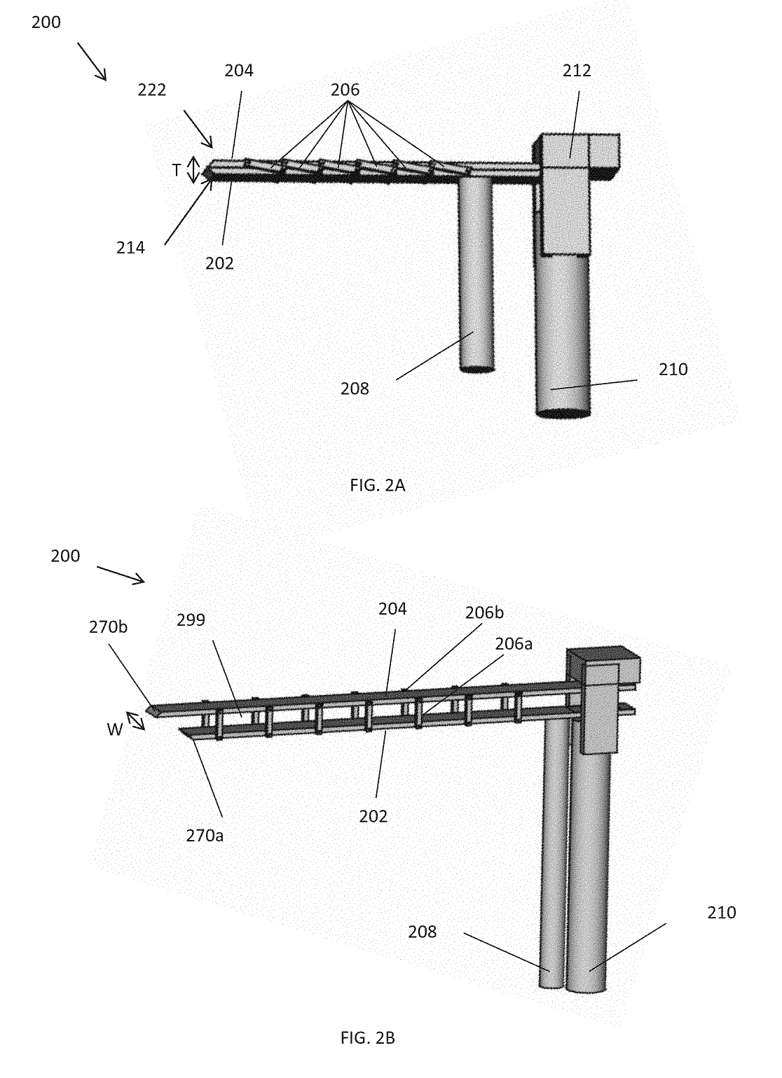

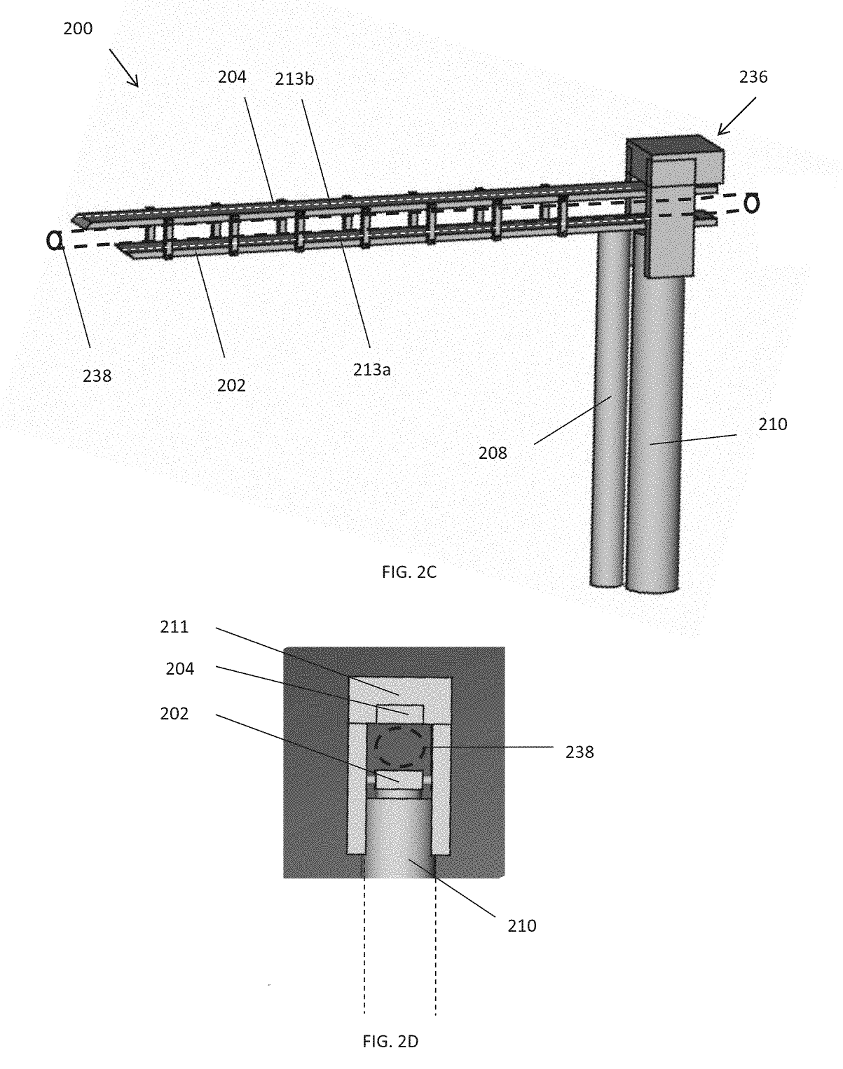

FIG. 2A is a simplified side view of an exemplary device 200, in a closed state, according to some embodiments of the invention.

FIG. 2B is a simplified side view of an exemplary device 200, in an expanded state, according to some embodiments of the invention.

FIG. 2C is a simplified side view of an exemplary device 200, after insertion of a tube 238, according to some embodiments of the invention.

FIG. 2D is a simplified top view of a proximal end 236 of an exemplary device 200, after insertion of a tube 238, according to some embodiments of the invention. Displayed in FIG. 2D are elongate elements 202, 204, where elongate element 202 is coupled to handle portion 210. In some embodiments, the device includes a housing 211, to which handle portion 210 is coupled. In some embodiments, housing 211 prevents lateral movement of elongate elements 202, 204 (e.g. movement perpendicular to movement in a direction of a long axis of the elements 213a, 213b).

### Exemplary Elongate Body and Elongate Element/s

### Exemplary Shape

In some embodiments, device 200 includes a body 222 which includes one or more elongated element 202, 204 which are pushed into the patient. In some embodiments, one or more elongated element has sufficient width, W, a measurement perpendicular to a long axis of the elongated element, to establish a sufficiently wide lumen for insertion of a drainage tube (e.g. 0.5-2 cm wide, or 0.8-1.5 cm wide, or lower or higher or intermediate ranges or widths).

In some embodiments, elongated elements 202, 204 are planar components where, for example, a long axis of one or more element is straight and/or a thickness of one or more element is constant along the length of the element. Alternatively, in some embodiments, the device body 222 is curved and/or bent, in one or more direction.

In some embodiments, in a closed (also herein termed collapsed) state, no lumen exists between first and second plates 202, 204. In some embodiments, when the device body is in a closed state, elongated elements 202, 204 are in contact with each other, e.g. for 90% or more of a length of device body 222.

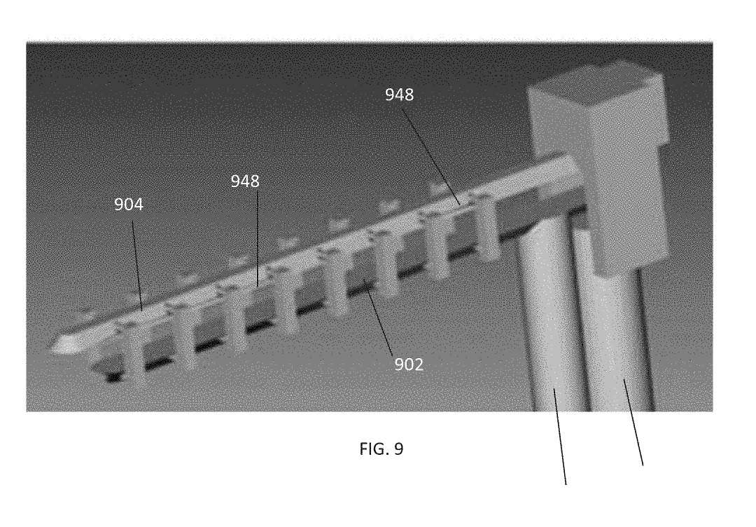

In some embodiments, elongated elements include a portion and/or shape protruding from a central axis of the body, for example one or more elongated element includes at least a portion of the elongated element including with a roof-shape cross section and/or a domed cross section (e.g. as illustrated in FIG. 9). In some embodiments, an elongated element including a cross sectional shape with a protrusion in a direction from the elongate body lumen, potentially eases (e.g. reduces the force required for) insertion of the elongate body (e.g. by moving tissue away from the body of the device).

In some embodiments, a length of the body of the device, measured from connection of the elongated elements to handles, e.g. a portion of the device for insertion and/or partial insertion into patient tissue is 1-40 cm or 1-30 cm or 1-25 cm or 2-25, or 10-25, or 15-25, or at least 1 or at least 2 or at least 5 cm long, or lower, or higher, or intermediate lengths or ranges.

### Exemplary Elongated Element Tip

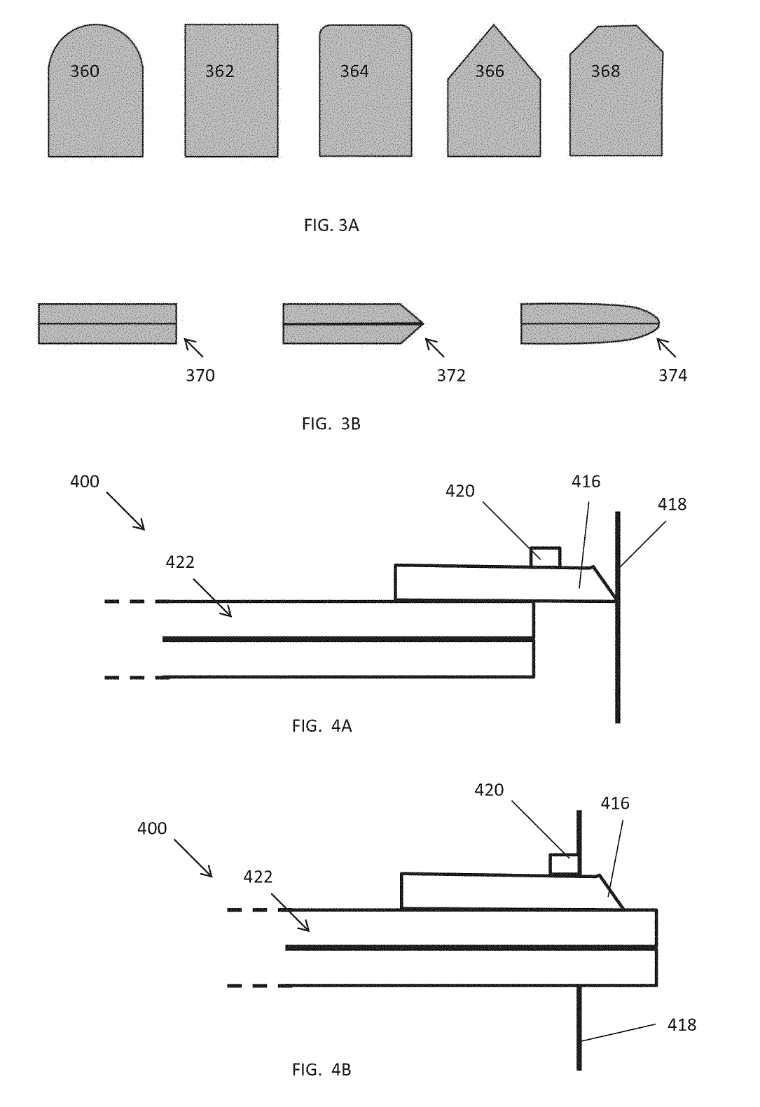

FIG. 3A is a simplified schematic top view of exemplary elongated element tips 360, 362, 364, 366, 368, according to some embodiments of the invention.

FIG. 3B is a simplified schematic side view of exemplary elongated element tips 370, 372, 374, according to some embodiments of the invention.

As mentioned previously, in some embodiments treatment starts by making an incision (e.g. using a scalpel) in the skin surface. In some embodiments, the device includes a blunt tip (e.g. elongated element/s include rounded tip/s), potentially reducing risk of damage during use of the device. In some embodiments, the device includes a blunt and/or rounded tip in one or more dimension.

For example, in some embodiments, a tip is rounded in one or more dimension. For example, a top view of tip 360 and a side view of tip 374, where, in both dimensions a tip edge does not include corners. In some embodiments, a blunt and/or rounded tip includes a tapered tip edge, for example, with an aspect ratio (length of contact of a tangent to the most distal part of the tip:length of tip taper) of more than 1:10, or more than 2:5, or lower or higher or intermediate ratios.

In some embodiments, an elongated element tip includes a tapered shape (e.g. 366, 360, 364, 368, 372, 374) in one or more dimension, for example, where a dimension of the tip is 1-80%, or 1-50%, or 1-20% or lower or higher or intermediate percentages or ranges of a size of the body of the elongated element in the same dimension. A tapered tip potentially reduces a force required to insert the device and/or assists in cutting of the skin surface with the device.

In some embodiments, a device includes a sharp tip, e.g. comprising a corner in one or more dimension and/or an aspect ratio of less than more than 1:10, or more than 2:5, or lower or higher or intermediate ratios in one or more dimension.

Referring now back to FIG. 2A and FIG. 2B, in an exemplary embodiment, tips 270a, 270b of the elongated elements 202, 204 are pointed parallel to the long axis and include a blunt top view.

In some embodiments, a body of a device includes a retractable and/or retracting sharp portion (e.g. knife). FIG. 4A is a simplified schematic side view of a portion of a device 400 including a knife 416 incising a skin surface 418 according to some embodiments of the invention. FIG. 4B is a simplified schematic side view of a portion of a device 400 including a retractable knife 416 after penetration by the device of a skin surface 418, according to some embodiments of the invention.

In some embodiments, after an initial incision is made in a patient's skin surface and/or once a desired depth of device penetration is reached, a user retracts knife 416, for example, by manually moving (and/or removing) the knife, e.g. by applying force to a knife handle 420. Alternatively, or additionally, in some embodiments, a user detaches knife 416, for example, by applying a force (e.g. pulling on) knife handle 420.

Alternatively, or additionally, in some embodiments, knife 416 self-retracts. For example, in some embodiments, knife 416 includes an attachment to a device body where the attachment releases upon insertion forces (e.g. reactive forces of the patient tissue on the knife). For example, in some embodiments, a knife is attached to the elongate body by a sliding attachment the friction of which is overcome by resistive forces of patient tissue after an initial incision. For example, in some embodiments, knife 416 retracts after the device is inserted to a certain depth insertion depth. For example, referring now to FIG. 4B, in some embodiments, handle 420 is a (e.g. blunt) element to which skin surface 418 applies a force, retracting knife 416.

### Exemplary Elongated Element Connection, Opening Mechanism

Referring back now to FIG. 2A and FIG. 2B, in some embodiments, movement of one elongated element with respect to another elongated element causes opening of a lumen between the elements.

In an exemplary embodiment, elongated elements 202, 204 are connected by one or more rigid portion 206, for example, in a direction perpendicular to a plane of the elongated elements. In some embodiments, rigid elements 206 transfer a direction of an applied force (e.g. by a user) into a force which expands elongate body 222 (e.g. enlarging and/or generating a lumen 299 therein).

In some embodiments, one or more rigid portions (e.g. rigid portions 206) are elongated cuboid elements, e.g. with square or rectangular cross section perpendicular to the long axis of the rigid portion. In some embodiments, rigid portions (e.g. rigid portions 206) each have the same geometry. In some embodiments, movement of elongated elements with respect to each other parallel to a long axis of the elements (e.g. movement of one or both elongated elements) causes the rigid elements to push the elongated elements apart in a direction perpendicular to the elongated elements long axes.

### Exemplary Easily Insertable Device, Exemplary Rigid Elements

In some embodiments, a body of the device (e.g. device 200) is easy to insert into and/or remove from tissue. In some embodiments, at least a portion of the device which is inserted into patient tissue includes a smooth and/or rounded outer shape when the device is in a closed and/or open state.

In some embodiments, the device includes an outer sheath and/or a sheath is placed onto the device before insertion of the device into a patient.

In some embodiments, rigid elements (e.g. rigid elements 206) do not protrude (or protrude at most by 0.1-3 mm, or 0.1-1 mm, or higher or lower or intermediate values or ranges) outside limits of the body (for example, the body including e.g. elongated elements 204, 206) of the device. Where outer limits of the device body are hereby defined as portions of the device body and/or planes connecting portions of the device body most removed from a lumen within elongated elements (e.g. lumen 299) and/or are defined as portion/s of the device and/or as planes connecting portions of the device which directly contact patient tissue during insertion of the device.

In some embodiments, rigid elements do not protrude (or protrude minimally, minimal protrusion hereby defined as protrusion of at most by 0.1-3 mm, or 0.1-1 mm, or higher or lower or intermediate values or ranges) for all outer limits of the device defined by the elongated elements, for example, in some embodiments rigid elements remain recessed within a space defined between the elongated elements for open and/or closed states of the device. In some embodiments, rigid elements do not protrude (or protrude at most by 0.1-3 mm, or 0.1-1 mm, or higher or lower or intermediate values or ranges) outside limits of the device defined by one or more outer plane/s of the elongated elements perpendicular to the elongated element long axes.

Referring now to FIG. 2A-C, in an exemplary embodiment, rigid elements do not protrude outside of elongated element outer planes perpendicular to long axes of the elongated elements and perpendicular to a direction of expansion of the space (e.g. lumen 299, e.g. direction of thickness, T) between the elongated elements when the device is opened.

In some embodiments, protrusion of rigid element/s is minimized and/or prevented by one or more of; position of attachment of the rigid elements, dimensions of the rigid elements (e.g. rigid elements 206 are sufficiently thin such that they do not protrude or protrude at most by 0.1-3 mm when the device is closed, e.g. as illustrated in FIG. 2A) and shape of the rigid elements. In some embodiments, rigid elements are internal to the outer limits of the elongated elements section of the device when the device is in an open and/or closed state. In some embodiments, rigid elements are aligned with the edges of the elongated elements section of the device when the device is in an open and/or closed state.

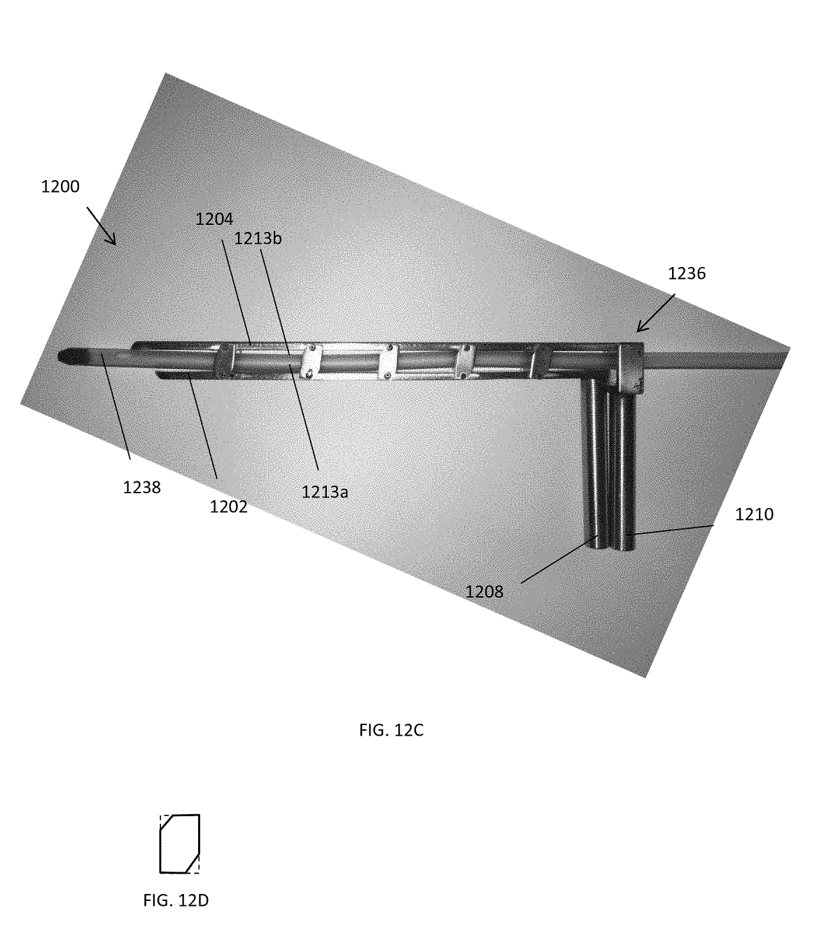

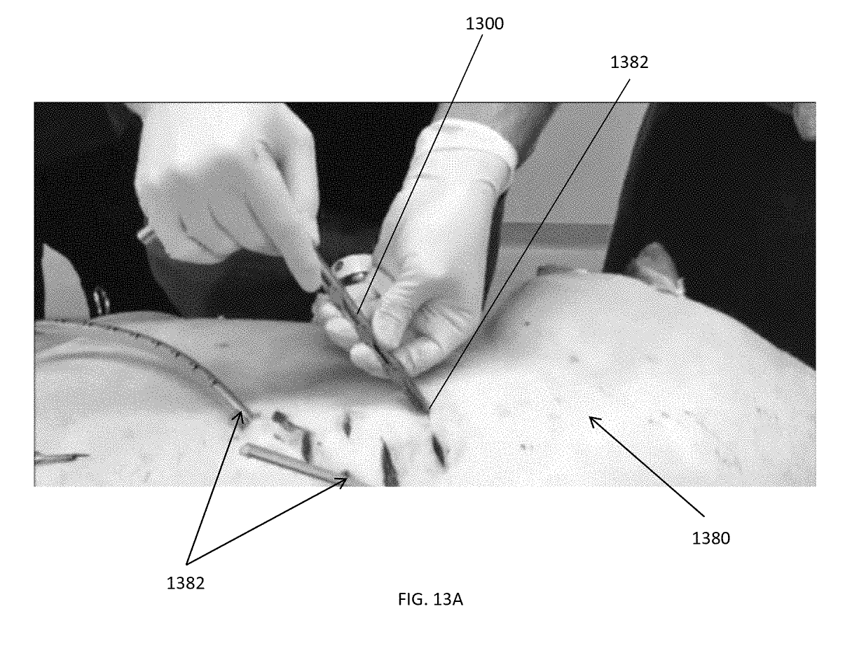

Referring now to FIG. 12A-C, in some embodiments rigid elements (e.g. rigid elements 1206 of a prototype device 1200) are a shape which is a rectangle with two or more triangular corner sections removed. FIG. 12D is a simplified schematic side view of a rigid element, according to some embodiments of the invention. FIG. 12D is a simplified schematic of a rigid element of the prototype device illustrated in FIG. 12A-C. Potentially, this shape enables a larger dimension of rigid elements parallel while maintaining low protrusion of the rigid elements. In some embodiments, a device includes alternative shapes for more or more rigid portions which do not protrude (or protrude minimally e.g. as defined above) e.g. when the device is in a closed state e.g. oval, rectangular with rounded corners, cylindrical struts.

A potential advantage of rigid elements 1206 with larger dimension parallel to the long axis of the elongated elements 1202, 1204 is increased strength and/or the ability to maintain strength of the elements while making the rigid elements small in a direction perpendicular to long axis of the elongated elements (e.g. protrude less).

In some embodiments, rigid elements 1206 are, for example, larger in comparison to rigid elements 206. In some embodiments, for example, one or more rigid element has length L1 (refer to FIG. 12B), where L1 is at least 1-30%, or 2-20%, or 2-10%, or 3-10% of a length of the device body, or lower, or higher, or intermediate percentages or ranges (length L FIG. 12A). In some embodiments, L1 is 1-50 mm, or 5-30 mm, or lower or higher or intermediate lengths or ranges.

For example, in some embodiments, a width of the rigid elements (dimension parallel to width W of the elongated elements is 0.1-10% or 0.1-5% or 1-5% or lower, or higher, or intermediate percentages or ranges, of a width of the elongated elements).

Lack of protrusions, for example, result in less friction when inserting the device into and/or removing the device from tissue.

### Exemplary Coupling of Rigid Element/s to Elongated Element/s

In some embodiments, connection of elongated elements is at an edge of the elongated elements, potentially minimizing a size of the inserted device within the body for a given lumen and/or channel and/or tube size.

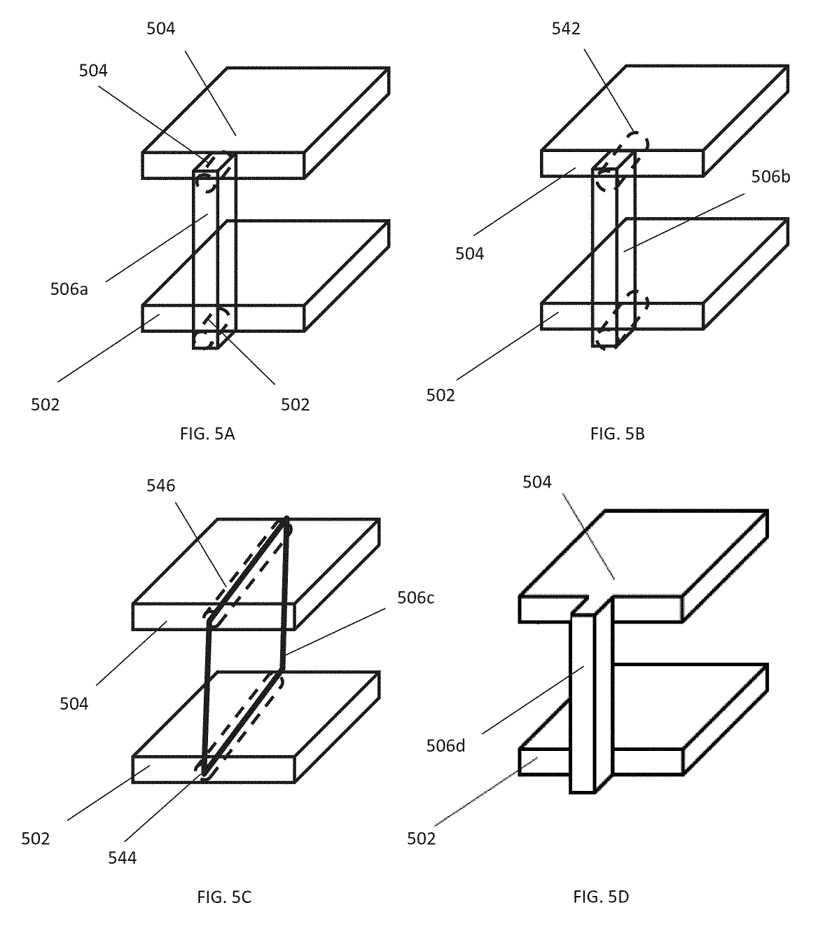

FIGS. 5A-5D are exemplary embodiments of a connection of a rigid portion 506 to elongated elements 502, 504, according to some embodiments of the invention.

In some embodiments, one or more rigid portion 506a-d is connected to elongated element/s by a pivot and/or hinge, for example, the rigid portion rotating around the pivot during expansion of the device. Referring now to FIG. 5A, in some embodiments, an elongated element includes one or more protrusion 540 which fits into a hollow within rigid element 506a. Alternatively, or additionally, in some embodiments, a rigid element includes a protrusion which fits into a hollow of an elongated element.

Referring now to FIG. 5B, in some embodiments, a separate component for example, a pin 542 connects rigid element 506b and one or more elongated element 502, 504, for example, by entering into hollows within the elements.

Referring back to FIG. 2A and FIG. 2B, in some embodiments coupling of elongated elements 202, 204 is at both sides of the elements. For example, the device including first rigid elements 206a and second rigid elements 206b.

In some embodiments, a rigid portion passes through a channel within one or more elongated element. For example, a single rigid portion coupling two sides of elongated elements. Referring now to FIG. 5C, in some embodiments, a rigid portion 506c passes through channels 544, 546.

In some embodiments, a portion elastically and/or plastically deforms in expanding the device. Referring to FIG. 5D, in some embodiments, a rigid portion 506d and one or more elongated element 504, 506 are one part (e.g. molded plastic), connection between the rigid part and the elongated elements, for example, being a living hinge and/or being deformed (e.g. plastically) during expansion of the body.

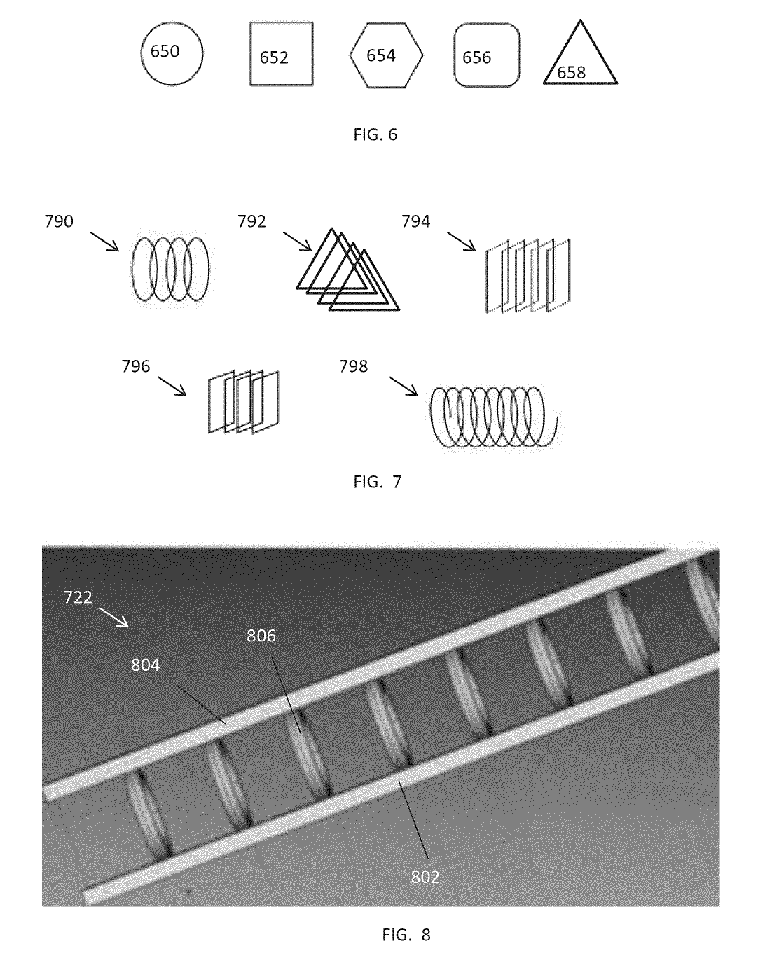

FIG. 6 is a simplified top view of exemplary shapes for rigid portions for connecting more than one elongated element, according to some embodiments of the invention. FIG. 7 is a simplified side view of exemplary pluralities of rigid portions, according to some embodiments of the invention.

In some embodiments, one or more rigid element has circular and/or oval shape e.g. 650, 790. In some embodiments, one or more rigid element has rectangular (e.g. square) shape, and/or a rounded rectangular shape e.g. 652, 656, 794, 796. Other exemplary shapes include triangle e.g. 658, 792 and polygons with more than 4 sides, e.g. 654 and irregular shapes.

In some embodiments, an element with a continuous shape (e.g. a helix 798) connects two or more elongated elements.

FIG. 8 is a simplified side view of an exemplary portion of a body 822 of a device including cylindrical shaped rigid portions 806, in an expanded state, according to some embodiments of the invention.

In some embodiments, one or more rigid portion (e.g. coupling the same sides of the elongated elements) is connected. FIG. 9 is a simplified schematic side view of a device including rigid portion connecting elements 948, according to some embodiments of the invention. In some embodiments, interconnection of rigid elements reduces the expanding force required to expand the device.

In some embodiments, two or more rigid portions are interconnected with an elastic element (e.g. elements 948 are elastic), the elastic element/s, for example, stretched when the device is in a closed state. In some embodiments, relaxing of the elastic element/s provides at least a portion of the expanding force required to open the device.

### Exemplary Expanding Force Mechanisms

Returning now back to FIG. 2A and FIG. 2B. In some embodiments, an expanding force is applied to a handle. In some embodiments, moving one or more handle portion moves one or more elongate element.

For example, in an exemplary embodiment, handle portions 208, 210 are pulled together (e.g. as illustrated in change of position of handle portions 208, 210 between FIG. 2A and FIG. 2B). In some embodiments, a first handle portion 208 is attached to a lower elongated element 202 and pulling handle portion 208 towards handle portion 210, moves lower elongated element 202 with respect to upper elongated element 204 (e.g. upper elongated element 204 is connected to second handle portion 210). Alternatively or additionally, in some embodiments, moving of a handle element moves upper elongated element 204.

In some embodiments, a handle portion directly applies a force to a portion of the device body (e.g. as illustrated in FIG. 2A and FIG. 2B). Alternatively or additionally, in some embodiments, a handle portion indirectly applies a force to a portion of the device body.

In some embodiments, the device includes a force multiplication element, for example, a lever and/or a gear, for example, in some embodiments, to multiply a force manually applied by a user, e.g. potentially increasing ease and/or speed of expanding of the device.

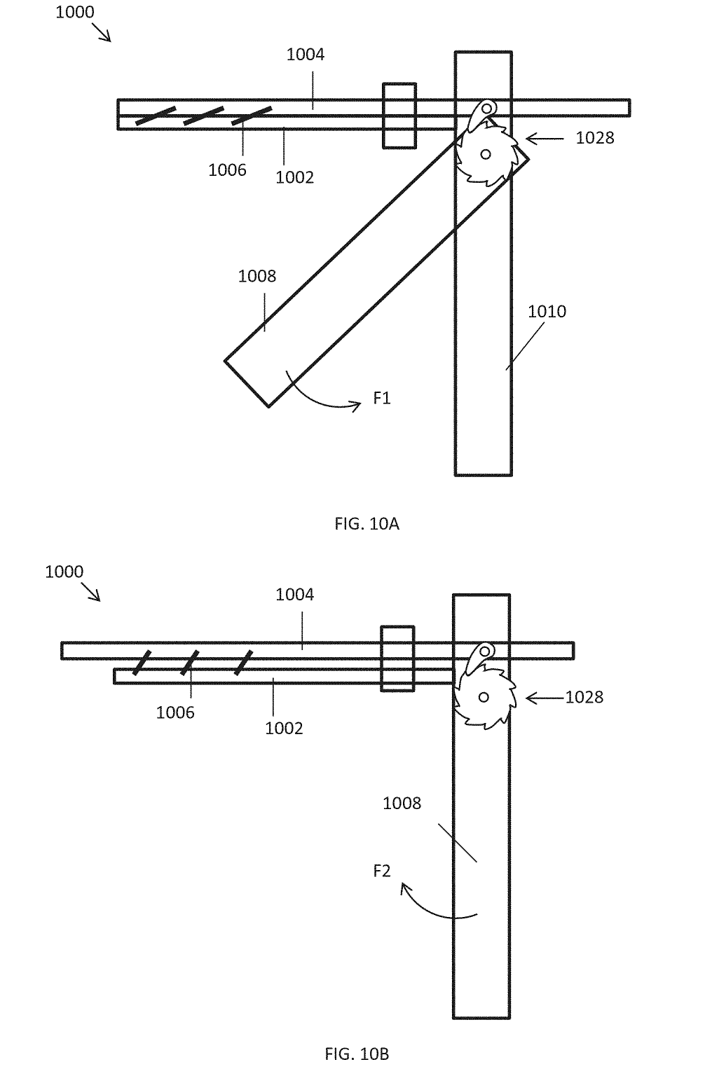

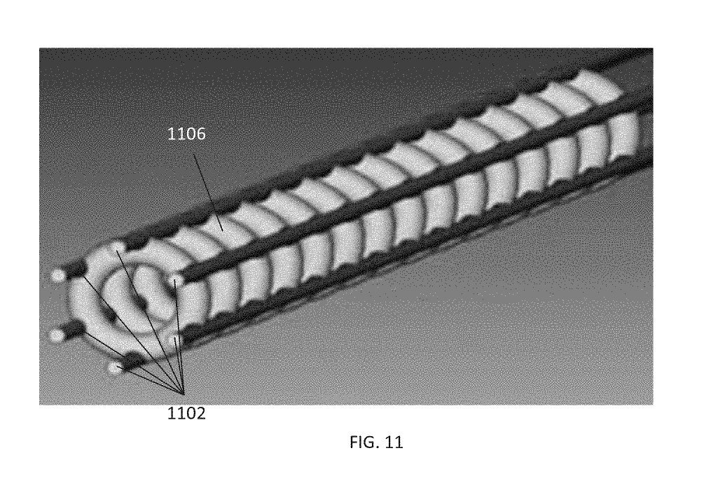

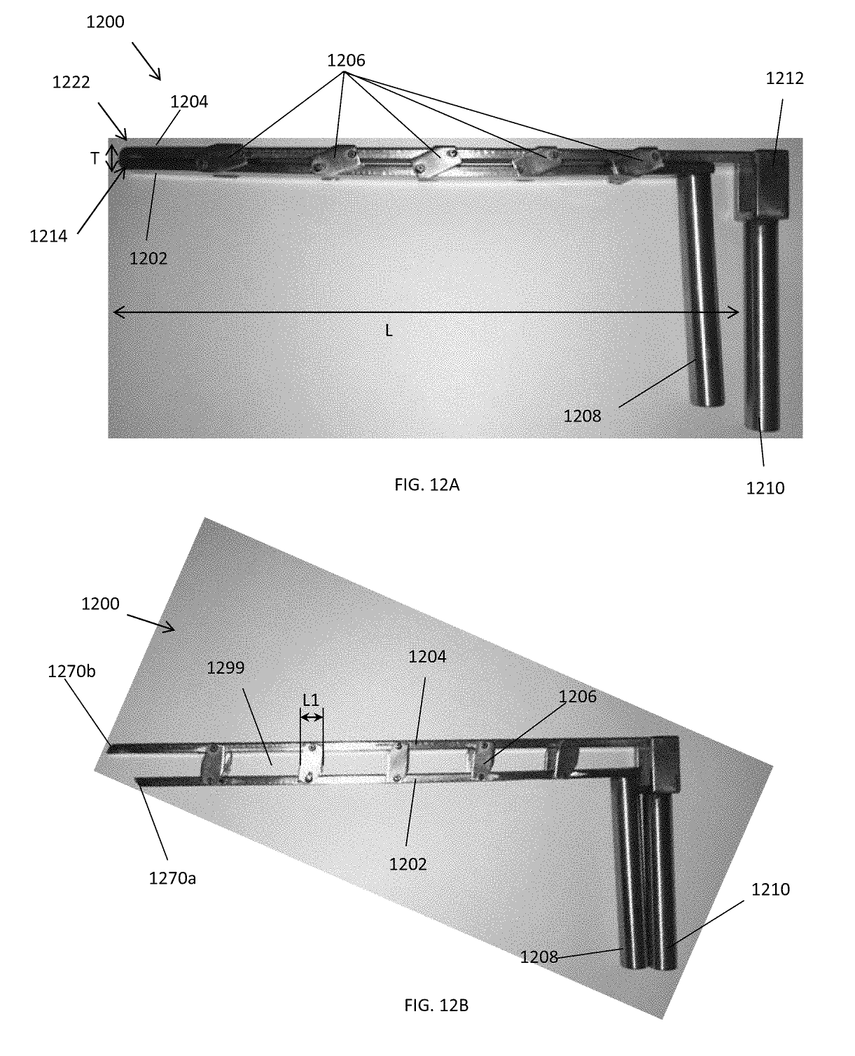

In some embodiments, expanding force is applied in stages and/or intermittently. FIGS. 10A-B are side views of a device 1000 including a ratchet mechanism 1028 which prevents retraction of a handle portion 1008, according to some embodiments of the invention.

FIG. 10A shows the device 1000 where the device is closed and elongated elements 1002, 1004 are in contact with each other. A force F1 is applied to a first handle portion 1008 bringing first handle portion 1008 from an initial position towards second handle portion and partially expanding a lumen between elongate elements 1002, 1004 as illustrated in FIG. 10B. In some embodiments, first handle portion 1008 is then moved back towards the initial position, by force F2. In some embodiments, force F2 is applied by a spring. To fully expand the device, the procedure as illustrated by FIG. 10A and FIG. 10B is repeated, in some embodiments, more than one time.

As mentioned elsewhere, in some embodiments, elastic force acts expand the device. For example, in some embodiments, two or more rigid portions are attached with elastic portions, where the elastic portions are under tension when the device is in a closed state. The device is either held and/or locked closed (e.g. by a locking element). Once released, the elastic elements relax to expand the device. Alternatively or additionally, in some embodiments, elastic element/s holding the device open and/or closed are located in other portions of the device, for example, the handle.

For example, returning to FIGS. 2A and 2B in some embodiments, one or more spring is coupled to handle portions 208, 210 is extended when the portions 208, 210 are separated (e.g. as illustrated in FIG. 2A). In some embodiments, the device is locked closed before insertion, and then released after insertion where the expanding force is applied by the spring/s.

In some embodiments, a torque and/or screw mechanism is used to expand the device, for example, a user applying torque to a handle portion. For example, in some embodiments, applied torque turns a screw mechanism to move one or more elongated element with respect to each other.

In some embodiments, the device includes one or more locking element which holds the elongate body open once it is expanded, the elongate body resisting collapse. For example, one or more lock and/or ratchet holds elongate body in an expanded state. For example, in some embodiments, rigid portions 206, once the elongate body is in an open configuration fall into hollows in the elongated elements 202, 206.

Exemplary Embodiments Including More than Two Elongated Elements

In some embodiments, a device body includes more than two elongated elements. FIG. 11 is a simplified schematic side view of a portion of a body of a device including more than two elongated elements 202, according to some embodiments of the invention. In some embodiments, a device includes 1-10, or 1-5 elongated elements one or more of which are moved to expand a lumen within the elongated elements. In some embodiments, a plurality of elongated elements 202 are connected by rigid portions 1106 with circular top view and rounded edges (e.g. toroid shape).

### Exemplary Materials

In an exemplary embodiment, elongated elements and rigid portions are constructed from metal, e.g. stainless steel. Alternatively, in some embodiments sufficiently strong (e.g. to expand the device within a patient without substantially bending) plastics are used for the elongated elements and/or rigid portions. In some embodiments, other portions (e.g. handle portions) of the device are constructed from stainless steel and/or plastic.

In some embodiments, a thickness of elongated elements is selected based on the material characteristics of the device and a thickness required to have bending of less than 0.01-1, or 0.01-0.1, or lower or higher or intermediate values or ranges, along the elongated element length, between rigid portions, during expansion of the device.

In some embodiments, at least a portion of the body includes biocompatible and/or low friction material (e.g. the body of the device is coated in the material). For example, potentially easing insertion and/or expansion of the device (e.g. reducing a force required to insert and/or expand the device and/or potentially reducing inflammation of surrounding tissue associated with use of the device.

### General

As used herein the term “about” refers to ±20%.

As used herein the term “approximately” refers to ±20%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### Exemplary Force Calculation

The force required to expand the device within a patient was estimated by using a modified combination pliers to generate a 1 cm diameter channel in a bovine rib meat cut using metal elongate elements about 3 mm thick, 1 cm wide and 5 cm long. The pliers were opened to expand the channel to 1 cm wide, by pulling on a spring scale which was used to measure the force required. The experiment was repeated 3 times between three different ribs of the bovine rib meat cut. Taking into account the leverage supplied by the pliers, the force needed to expand the channel was 150-180N.

### Exemplary Implementation

A prototype device was constructed out of stainless steel. FIG. 12A is a side view of a prototype device 1200, in a closed state, according to some embodiments of the invention. FIG. 12B is a side view of a prototype device 1200, in an expanded state, according to some embodiments of the invention. FIG. 12C is a side view of a prototype device 1200, after insertion of a tube 1238, according to some embodiments of the invention.

The prototype device 1200 was built for suitability with an approximately 0.8 cm diameter tube. Approximate dimensions of the prototype: Length, L was approximately 20 cm. Width (refer to “W”, FIG. 2B) of the elongated portions was approximately 1 cm. Anterior handle length 1210 was approximately 7 cm, Posterior handle length 1208 was approximately 6.3 cm.

In some embodiments, a tube for use with human patients is approximately 1 cm in diameter corresponding, in some embodiments, to a larger dimensioned device, e.g. a larger width (e.g. 1.1 cm-3 cm or 1.1-1.5 cm) device.

An exemplary device, as illustrated in FIGS. 12A-C was tested three times on a recently euthanized pig.

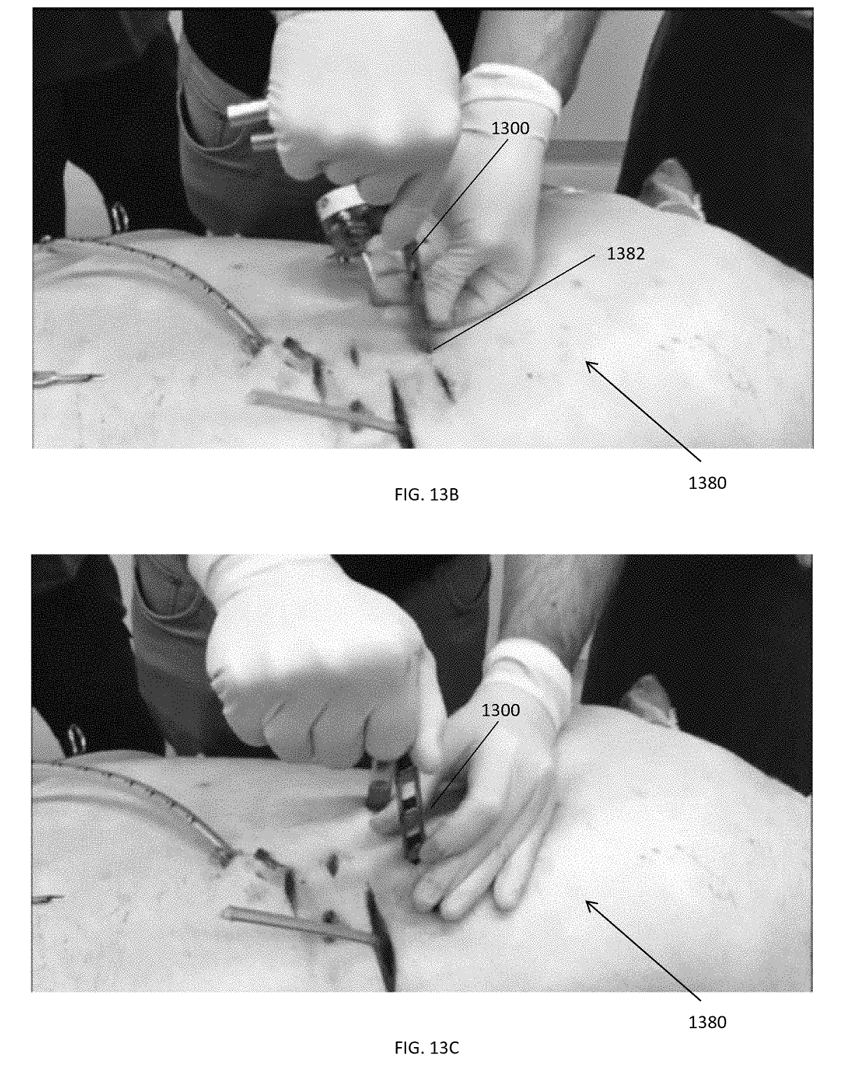

Testing involved making a small incision (approximately 2-3 cm in length) in the skin between two ribs. A device in a closed position was then positioned at the incision. FIG. 13A is an illustration of an outside view of a pig when a device 1300, in a closed state, is positioned at an incision site 1382 on the torso of a euthanized pig 1380, according to some embodiments of the invention. FIG. 13A illustrates the third test, tubes 1384 from the first two tests are visible, having been left remaining in situ after the test, according to some embodiments of the invention.

By blunt dissection, the devise was then inserted through the incision until the device reached the pleural cavity. The force required for insertion of the device to the pleural cavity was found to be reasonable. No damage to the tissues or the lung was noted. FIG. 13B is an illustration of an outside view of the pig when the device, in a closed state, has been inserted sufficiently to penetrate the pleural cavity, according to some embodiments of the invention.

The device 1300 was then opened. FIG. 13C is an illustration of an outside view of the pig after opening of the device 1300, according to some embodiments of the invention. Opening of the device was found to require minimal force. Once the device was opened, a chest tube was inserted through the device, and the device was removed, leaving the tube in place.

Removal of device 1300 in the open state was found to be more difficult, the users suggested a possible cause for this being friction associated with edges of the prototype e.g. edges of the rigid elements and/or protrusion of the rigid elements. A potential benefit of a device including protrusions when the device is (e.g. of rigid elements) and/or increased friction is that the device remains in situ e.g. during insertion of a tube and/or if the patient moves and/or is moved.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of accessing a thoracic cavity;
inserting, in an insertion direction, a thin rigid elongate expandable body, from outside the body, through a space between adjacent ribs, to within the thoracic cavity;
expanding said expandable body in a direction including a component perpendicular to said insertion direction to generate an expandable body lumen therein.

2-41. (canceled)

42. A device for establishing a channel to a thoracic cavity from outside a body comprising:
an elongate rigid expandable body, at least 2 cm long, configured to have collapsed state and an expanded state, said body in said expanded state comprising a lumen running through a length of said expandable body;
a handle coupled to said elongate expandable body;
an expander which transfers a force applied to said handle into an expanding force to expand said expandable body.

43. The device of claim 42, wherein said device forms part of a kit, said kit comprising a tube sized to fit into said lumen.

44. The device of claim 43, wherein said tube is lubricated.

45. The device of claim 42, wherein a lumen of said elongate expandable body is lubricated.

46. The device of claim 42, wherein said handle comprises a first part which moves relative to a second handle part, said handle parts interconnected by at least one mechanical gain element which transfers force applied to said handle parts to said expander.

47. The device of claim 46, wherein said at least one mechanical gain element comprises a lever.

48. The device of claim 46, wherein said at least one mechanical gain element comprises a gear.

49. The device of claim 42, wherein a ratchet connects said expander and said expandable body; wherein said ratchet prevents retraction of said expander.

50. The device of claim 42, wherein a ratchet connects at least one handle portion to said body; wherein said ratchet prevents retraction of said handle.

51. The device of claim 42, comprising an elastic element coupled to said expander, which elastic element is elastically deformed when said device is in said collapsed state and elastically relaxes applying an expanding force to said expander.

52. The device of claim 42, wherein said body comprises a plurality of elongated elements;
wherein said expander comprises at least one rigid element coupling said elongated elements;
wherein said expanding force moves said elongated elements with respect to each other;
wherein said at least one rigid element transfers a direction of the force of said elongated elements moving with respect to each other into an expanding force: said movement of said elongated elements changing an angle of said at least one rigid element with respect to said elongated elements, expanding a separation between said elongated elements.

53. The device of claim 42, wherein said body comprises two elongated elements, said elongated elements orientated with parallel long axes;
wherein said expanding force moves the elongated elements with respect to each other in a direction parallel to the long axes of the elements.

54. The device of claim 42, wherein said body includes a sharp tip positioned for penetrating said the chest outer surface.

55. The device of claim 54, wherein said sharp tip is retractable.

56. The device of claim 42, comprising a cover covering an opening of said lumen.

57. The device of claim 42, comprising a stopper attached to or disposed on said expandable body and increasing a geometry of said expandable body in a direction of expansion of said expandable body, said stopper resisting insertion of said expandable body into the thoracic deeper than the location of said stopper of said body.

58. The device of claim 57, wherein said device comprises a plurality of stoppers, where one or more stopper is collapsible into a state where the stopper protrudes from said expandable body by at most 3 mm.

59. The device of claim 42, wherein said device comprises a locking element configured to hold said elongate body open in said expanded state.

60. The device of claim 42, wherein one or both of said elongated elements has a tapered tip wherein a dimension of said tip is 1-80% of a size of a body of said elongated element in the same dimension.

61. The device of claim 42, wherein said body comprises at least two elongated elements;
wherein said expanding force moves the elongated elements with respect to each other in a direction including a component perpendicular to a long axis of at least one of said at least two elongated elements.
